(19) United States
(12) Patent Application Publication
(10) Pub. No.: US 2002/0197633 A1
(43) Pub. Date: Dec. 26, 2002

(54) Evaluation of ultraviolet radiation damage to skin using new gene markers, methods and compositions related thereto

(76) Inventors: Brian C. Jones, Warwick, NY (US); Carol Bosko, Oradell, NY (US); Kevin Cooper, Moreland Hills, OH (US); Thomas McCormick, Orange Village, OH (US)

Correspondence Address: MORGAN & FINNEGAN, L.L.P., 345 Park Avenue, New York, NY, 10154-0053, US

(21) Appl. No.: 10/143,360

(22) Filed: May 9, 2002

Related U.S. Application Data
(60) Provisional application No. 60/289,680, filed on May 9, 2001.

(51) Int. Cl. C12Q 1/68; C07H 21/04; C12N 15/01

(52) U.S. Cl. 435/6; 435/448; 536/23.2

(57) ABSTRACT

The present invention describes a method for treating and/or evaluating photodamage and/or photoaging of skin caused by exposure to solar ultraviolet (UV) radiation. The method employs a unique set of marker genes whose expression was newly found to be altered following exposure of skin to UV radiation. The invention provides an advantageous system of identifying and assessing substances that are capable of modulating, e.g., via attenuation, UV radiation induced alteration or change in the expression of at least one of the newly provided marker genes in skin relative to the gene expression level in skin not exposed to UV radiation. Also provided are compositions comprising materials that upon application to skin can modulate the gene expression of at least one gene of the marker gene set after exposure of skin to UV radiation, thereby affording protective and therapeutic effects and treatments for photodamage and photoaging. The potential benefit of, e.g., skincare, hair care, cosmetic, and personal care agents, and nutritional supplements, as materials having antiphotodamage and/or antiphotoaging properties can be assessed using the present method.

This application claims benefit of provisional application U.S. Serial No. 60/289,680, filed on May 9, 2001, the contents of which are hereby incorporated by reference in their entirety.

### FIELD OF THE INVENTION

The present invention generally relates to treatment and protection of human skin from photodamage and photoaging. More particularly, the present invention relates to the prevention, treatment, amelioration and repair of damage to skin caused by exposure to ultraviolet (UV) radiation (photodamage) utilizing novel gene markers. The invention further relates to methods for preventing, treating, ameliorating and reversing photoaged skin by the use of materials affecting a set of previously unidentified marker genes whose expression has been newly found to be associated with exposure of skin to UV radiation. The invention further relates to methods for evaluating and assessing UV radiation damage to skin by providing a set of previously unidentified marker genes whose expression has been newly found to be associated with exposure of skin to UV radiation. In addition, the invention relates to compositions, preferably for topical application, to protect against, ameliorate, prevent, inhibit, block, reduce, treat, or reverse photodamage and photoaging of skin, especially as induced by acute and chronic exposure to incidental and/or direct UV radiation, such as occurs daily and over time.

### BACKGROUND OF THE INVENTION

Sunlight, especially UV radiation, damages human skin, causing, among other undesirable effects, photoaging, immunosuppression and skin cancer.

Human skin comprises two compartments: a superficial outer compartment, the epidermis, and a deeper compartment, the dermis. Although the outermost epidermal skin layers typically provide a certain degree of protection to the body, the epidermis and dermis bear the brunt of the above-noted harmful effects of photodamage. The natural human epidermis is mainly composed of three cell types, namely, the keratinocytes, which are highly predominant, the melanocytes, and the Langerhans' cells. These cell types function to provide the essential protectant role of the skin in the human body. The dermis, which provides a solid and nutritional support to the epidermis, comprises mainly fibroblasts and an extracellular matrix composed primarily of collagen, elastin and a ground substance, which are synthesized by fibroblasts. In addition, the dermis contains leukocytes, mastocytes, tissue macrophages, blood vessels and nerve fibers.

Solar radiation is known to comprise ultraviolet (UV) (.lambda.<400 nm), visible (400 nm<.lambda.<700 nm), and infrared (IR) (.lambda.>700 nm) radiation. UV radiation is generally divided into UVA (320-400 nm), UVB (290-320 nm), and UVC (<290 nm). UVC radiation is generally blocked from reaching the earth's surface by stratospheric ozone. It is the ultraviolet (UV) component of sunlight, particularly UVA and UVB, that is generally believed to be the principal causative agent in photoaging and photodamage. With respect to the human body, the skin epidermis is the first target to be reached by solar radiation, in particular, UV radiation.

The extent of UV radiation exposure that is required to cause photoaging and/or photodamage to human skin is not currently well defined, although the amount required to cause erythema (reddening), commonly manifested as sunburn, in human skin is known and is quantified empirically as the “minimal erythemal dose” (“MED”) from a given UV source.

Exposure of the skin to solar radiation, especially UV radiation, can lead to changes in the skin and in the content of certain compounds within the skin, thereby accelerating the natural skin aging process. The process of accelerated or premature aging of the skin due to UV radiation exposure is generally called photoaging (also, actinic aging or dermatoheliosis).

Photoaging results from the action of extrinsic factors and effects on the skin, including solar radiation, particularly UV radiation. The phenotypic effects of photoaging on skin are typically characterized clinically by a loss of elasticity, coarseness, mottled pigmentation, sallowness, laxity, a dry, rough appearance associated with elasticity loss, a change in the pores of the skin and both superficial and deep wrinkles, particularly around the eyes. Frequently, premalignant and malignant neoplasms are associated with repeated sun exposure and photoaging. Photoaging commonly occurs in skin that is habitually exposed to sunlight, such as the face, ears, bald areas of the scalp, neck, torso, arms (e.g., forearms), legs, feet and hands.

Sunscreens are commonly used to prevent photodamage and photoaging of skin areas that are exposed to sunlight. Sunscreens are topical preparations that contain ingredients which absorb, reflect, and/or scatter UV light. Some sunscreens are based on opaque particulate materials, e.g., inorganic materials, or a combination of inorganic and organic materials, including zinc oxide, titanium oxide, clays, and ferric chloride, which produce a visible protective layer. Other sunscreens contain components to yield a transparent or translucent product on the skin. Compounds comprising sunscreens include, but are not limited to, oxybenzone, sulisobenzone, dioxybenzone, menthyl anthranilate, para aminobenzoic acid (PABA), octyl methoxycinnamate, octocrylene, drometrizole trisiloxane, octyl salicylate, homomenthyl salicylate, octyl dimethyl PABA, TEA salicylate, titanium dioxide, zinc oxide, butylmethoxy dibenzoylmethane, 4-methyl benzilidene camphor, octyl triazone, terephthalydiene dicamphor sulfonic acid, ethyl PABA, hydroxy methylphenyl benzotriazole, methylene bis-benzotriazoyl-tetramethylbutylphenol, bis-ethylhexyloxyphenol methoxyphenol triazine and mixtures of the foregoing. Other suitable and useful sunscreen actives include those disclosed in U.S. Pat. No. 5,000,937 to J. F. Grollier et al.

The development of the extrinsic effects of UV radiation exposure on skin depends upon a number of complex gene interactions and feedback mechanisms that can result in generally unpleasant or more severe pathological phenotypic changes, for example, premature wrinkling and/or skin cancer. Such complex genetic interactions have been previously quite difficult to ascertain and understand as a consequence of limitations in technology and the labor-intensive nature of prior methods.

Several current methodologies for evaluating UV-induced skin damage rely on the generation and measurement of erythema (i.e., skin redness). The erythemal response is quite variable from one individual to another, as it is dependent on genetic makeup of the individual, including skin type, ethnic background, and the like. Further, the endpoint is highly subjective and significant damage, such as cellular damage, can occur in the absence of visible erythema.

Other methods for determining UV-induced damage or aging of skin do not enlist molecular procedures to identify and assess the UV radiation induced altered expression of a newly discovered set of genes, as does the present invention. In addition, the newly-discovered genes that signal UV damage effects in in vivo human skin have not been previously identified as such by other methods. For example, U.S. Pat. No. 6,018,098 to Bernstein et al. discloses in vivo and in vitro models of cutaneous photoaging in which a reporter gene for the elastin promoter is employed to assess activation of the elastin promoter reporter gene upon UV radiation exposure. U.S. Pat. No. 6,130,254 to Fisher et al. discloses a method involving the enzyme activation of matrix metalloproteinases (MMP) to assess damage following UV radiation exposure. U.S. Pat. No. 6,079415 to Bernerd et al. discloses a process for evaluating damage to skin after type A ultraviolet radiation exposure in which a variation to a marker specific for the type A UV-induced skin damage is measured in an in vitro skin equivalent. Such marker variations include type 1, or interstitial, collagenase; vimentin analysis and unspecified variations in cells, nucleic acid, protein, ions, organelles, lipids and polysaccharides. U.S. Pat. No. 5,691,158 to Reece et al. discloses a tissue model, i.e., an artificial skin culture, to determine the efficacy of sunscreen formulations by induction of an inflammatory mediator, e.g., interleukin-1-alpha, or viability by cytotoxicity assay (e.g., MTT).

With the advent of the tools of molecular biology, particularly, microarray analysis, complex genetic changes and interactions have begun to be elucidated. The present invention utilizes novel gene markers, measurable by a number of convenient molecular tools and parameters, preferably via nucleic acid array technology, to evaluate photodamage to, and/or photoaging of, skin resulting from UV radiation exposure. As described herein, novel and advantageous methods and compositions are provided by the present invention.

### SUMMARY OF THE INVENTION

The present invention provides a method of evaluating damage to the skin caused by solar/UV radiation, i.e., photodamage. As used herein, “UV radiation” includes “solar radiation”. The method involves the assessment of changes or alterations in gene expression and/or activity of one or more, or a combination of, genes identified and described herein, following exposure to UV radiation. In accordance with the present invention, a unique and unexpected set of genes has been determined for use in the method due to alteration in the expression of one or more of these genes following exposure of skin to UV radiation. A specific aspect of the method involves obtaining nucleic acid, e.g., RNA, from a skin source that has been exposed to UV radiation, and assaying the RNA to determine if there is UV radiation-induced altered expression of one or a combination of the gene markers that have been newly discovered to exhibit altered expression after UV radiation exposure, relative to non-UV radiation exposed skin. Microarray analysis of the RNA is preferred in this specific aspect of the invention.

It is another aspect of the present invention to provide a method of evaluating compositions that modulate, e.g., by attenuation, or effect modification of gene expression associated with UV radiation exposure of skin. Such compositions include, without limitation, topically applied sunscreens, anti-oxidants, anti-inflammatories, cosmetics, including makeups, nutritional supplements and other systemic oral agents, anti-aging formulations, e.g., creams for fine lines and/or wrinkles, topicals, skin permeants and the like. Also in accordance with this invention, ingredients, components, or compounds that are formulated in such compositions in a variety of product forms, e.g., transdermals, such as patches, and the like, are evaluated for their photoprotective effects by assessing their ability to modulate of prevent gene expression associated with UV radiation exposure of skin. Such compositions, ingredients, components, compounds, and/or products are intended for both topical and oral administration. The evaluation method employs one or more of the novel set of gene markers that have been discovered to have altered expression after UV radiation exposure of skin as described herein.

It is another aspect of the invention to provide a new means to test and evaluate substances, e.g., components or ingredients of anti-aging or anti-photodamage formulations, such as sunscreen formulations or products, or nutritional supplements, to identify those substances that ameliorate, treat, prevent, inhibit, block, reduce, or repair UV radiation-induced damage to skin, and/or that provide photoprotection to UV radiation damage, and/or that ameliorate, treat and/or reverse photoaged skin. In accordance with the present invention, such substances that can ameliorate, treat, prevent, inhibit, block, reduce, repair, or reverse UV radiation induced skin damage are suitable for use as ingredients in cosmetics, sunscreens, anti-aging products, or dermatological nutritional supplements, for example, to yield photoprotection and/or anti-aging protection (i.e., protection against UV radiation induced damage) to the user, preferably to the skin of the user.

It is yet another aspect of the present invention to provide a new method for determining if a product under development, e.g., a sunscreen formulation or a prototype thereof, possesses a desired level of protection from UV radiation exposure. Thus, the present invention provides a method of screening for a desired sun protection factor or level thereof for a sunscreen or anti-aging composition by determining if the factor or level thereof modulates the expression, relative to control, of at least one of the genes comprising the new marker gene set whose expression is altered as a result of exposure to UV radiation.

Another aspect of the present invention provides materials which can modulate gene expression in unprotected skin and/or reverse the expression levels of at least one gene of the set of marker genes whose expression is altered following exposure of skin to ultraviolet radiation compared with control, e.g., unexposed skin. Exposure to UV radiation results in photodamage and/or photoaging of skin. The present invention relates to materials that are able to ameliorate, treat, prevent, inhibit, block, reduce, repair, and/or reverse photodamage, and/or ameliorate, treat, prevent, inhibit, block, reduce, repair, or reverse photoaging of skin. In a related aspect, the invention also provides compositions and formulations comprising one or more of the above-described materials, particularly in an amount effective to modulate or reverse the expression of at least one of the marker genes as described herein. Such compositions and formulations, and the materials therein, are useful for treatments or protectants for photodamage and photoaging of skin, including unprotected skin, skin tissue, other skin equivalent or keratin forming cells.

In another of its aspects, the present invention provides a method of evaluating the ultraviolet radiation protective, therapeutic and/or treatment effects of a compound or component to photodamage or photoaging of skin. The method comprises contacting the compound or component undergoing evaluation with a test material selected from skin or a skin substitute, e.g., skin equivalent, skin cells, or keratin forming cells, and exposing the test material to an ultraviolet radiation source. Thereafter, it is assessed, using methods practiced in the art, whether the compound or component modulates the expression levels of at least one gene of a set of marker genes of the test material following exposure to ultraviolet radiation compared with a control, e.g., test material unexposed to UV radiation.

In another aspect, the present invention provides a method of ameliorating or treating photodamaged or photoaged skin. In particular, the present invention provides a method of repairing photodamaged skin or reversing photoaged skin. The method involves applying to skin, or an area thereof, a composition containing a material that modulates the expression of at least one gene of a set of marker genes whose expression is altered following exposure of the skin to ultraviolet radiation. The composition is preferably topically applied in an amount and for a period of time effective to modulate the expression of at least one gene of the novel marker gene set of the present invention following exposure to ultraviolet radiation.

According to inventive method of this invention, the ability of a composition, and/or the active material formulated therein, to modulate expression levels of at least one of the marker genes upon application to skin and following UV radiation indicates that the composition is capable of ameliorating, treating, repairing or reversing photodamaged or photoaged skin exposed to UV radiation. The modulation of expression of the at least one gene in the marker gene set by the composition is typically compared with at least one control, e.g., the expression of the genes in skin, or a skin substitute, that has not been exposed to UV radiation.

Another aspect of the present invention provides a composition, preferably a cosmetic composition, containing one or more photoprotective, phototreatment, and/or anti-photoaging components, materials, or substances, wherein the one or more components, materials, or substances have been demonstrated to provide photoprotection, phototreatment, or anti-photoaging effects, by ameliorating, inhibiting, blocking, reducing, preventing, repairing, treating, or reversing UV radiation-induced damage to, or aging of, skin as assessed by the ability of such components, materials, or substances to modulate the alteration of expression of one or more marker genes newly found to be associated with exposure to UV radiation. Preferably, modulation by the component, material or substance results in expression of the one or more marker genes that reflects, or is similar to, the level of gene expression in controls that have not been exposed to UV radiation.

Yet another aspect of the present invention provides a kit containing a support or support material, e.g., a membrane, more particularly, a nitrocellulose or nylon membrane, comprising the novel set of genes whose expression is altered upon exposure of skin to UV radiation in a form suitable for the practitioner to employ in identifying compounds, materials, reagents, ingredients, agents, and the like which have a photoprotective effect and/or which can ameliorate, repair, prevent, inhibit, block, reduce, treat, or reverse photodamage and/or photoaging caused by UV radiation exposure of skin, skin substitute, skin cells, or keratin forming cells. In addition to the gene set which provides an identified target set of gene markers for determining those agents that are able to overcome, or otherwise affect, UV radiation-induced damage and/or aging, the kit can contain other materials necessary for carrying out the assay method, including but not limited to, labeled probes, buffers, controls, and instructions for use.

In yet another aspect, the present invention provides the identification of individuals, i.e., a subset of individuals, who respond to UV induced gene expression by having altered expression of one or more of the marker genes in the UV radiation-induced marker gene set as newly-described herein. Such individuals would be more susceptible to photodamage and photoaging following exposure to UV radiation. These individuals, who could be identified through screening using the methods of the present invention, would be especially amenable to treatment or therapies involving materials, ingredients, compounds, formulations and compositions that are capable of modulating an alteration in the expression of one of more genes of the marker gene set as described herein. The identified individuals would be particularly suitable to amelioration, reduction, treating, preventing, repairing, reducing, and or reversing of UV radiation induced photodamage and photoaging.

In accordance with each of the foregoing aspects of the present invention, the marker gene set, which comprises at least one gene whose expression is altered after UV radiation exposure, include Ras related protein RAB-7; Corneodesmosin; Amphiregulin; Granulocyte Chemotatic Protein; Migration inhibitory factor MRP8 (calgranulin A); Migration inhibitory factor MRP14 (calgranulin B); Ephrin receptor; epithelial cell kinase (ECK); shb proto-oncogene; MAD transcriptional repressor; Calpain; Leukocyte elastase inhibitor (monocyte/neutrophil elastase inhibitor); Placental plasminogen activator inhibitor (PAI-2); Beta-defensin (human beta defensin 2, HBD2 and human beta defensin 3, HBD3); Alpha 1 antitrypsin precursor; Tristetraproline; growth factor inducible nuclear protein 475; Interferon regulatory factors (IFR family); Nuclear Factor 1; hSNF2 Transcriptional activator; Prothymosin; GATA3 transcription factor; Histidine decarboxylase; Acyl Co-A binding protein; Decorin; CD44 antigen; B94 protein; Transthyretin (TTR), (prealbumin); Apolipoprotein E; Epithelial discoidin receptor; Thrombin receptor; serine/threonine protein phosphatase; Leukocyte antigen-related protein (LAR); Cytochrome p450 IVB1; Thioredoxin peroxidase; Myristylated Alanine-Rich C Kinase Substrate, MacMARCKS (MRP); EB1 microtubule associated protein; a unique subfragment of the marker genes, and combinations thereof.

The ability of a material, compound, component, ingredient, substance, or composition to modulate the expression level of at least one gene of the above listed marker gene set relative to control (e.g., skin not exposed to UV radiation, or skin whose exposure to UV radiation has been blocked or attenuated) correlates with the photoprotective and therapeutic ability of the material to ameliorate, treat, prevent, inhibit, block, reduce, and/or repair photodamage, and/or ameliorate, treat, prevent, inhibit, block, reduce, and/or reverse photoaging of skin.

Further aspects, features and advantages of the present invention will be better appreciated upon a reading of the detailed description of the invention when considered in connection with the accompanying figures/drawings.

### BRIEF DESCRIPTION OF THE FIGURES

FIGS. 1A and 1B present representative cDNA array images of the expression pattern of genes in normal control skin (FIG. 1A) and solar stimulated radiation (SSR)-exposed skin 32 hours after exposure to 4 minimum erythema dosage units (4 MED) of UV radiation, (FIG. 1B).

FIGS. 2A and 2B show a scatter plot analysis of log-transformed gene expression data for two different experiments from the same pooled skin samples. Each point represents the normalized expression level of an individual gene. The line indicates a best fit regression.

FIG. 3 presents a scatter plot analysis of long-transformed gene expression data for exposed skin versus normal (unexposed) control. Each point represents the average of the normalized expression level of an individual gene from four experiments. The lines indicate the confidence intervals equal to about two standard deviations (±0.35).

FIGS. 4A and 4B show the results of reverse transcription PCR (RT-PCR) performed to confirm the up-regulation of one of the gene markers, human beta defensin 2 (HBD2) and human beta defensin 3 (HBD3) as described by the present invention.

FIG. 4A: HBD2 gene expression following SSR of human skin (RT-PCT of pooled RNA).

FIG. 4B: HBD3 gene expression following SSR of human skin (RT-PCT of pooled RNA).

FIGS. 5A and 5B show a RT-PCR time course analysis of HBD2 and HBD3, following SSR exposure of human skin. In these analyses, keratomes were taken from non-irradiated skin and from skin at 8, 24, 32, or 48 hours following 4 MED SSR. RNA was extracted from keratome samples; RT-PCR was performed for HBD2 and HBD3. (FIG. 5A), (see also, Example 1). Normalized relative fold increase is shown in FIG. 5B.

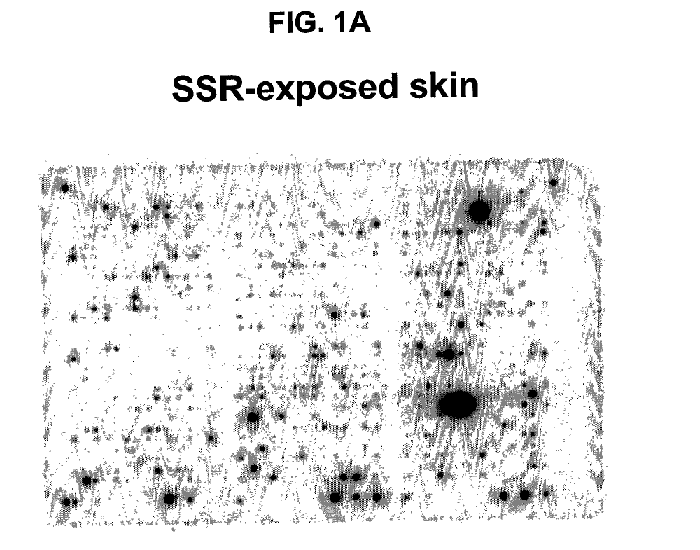

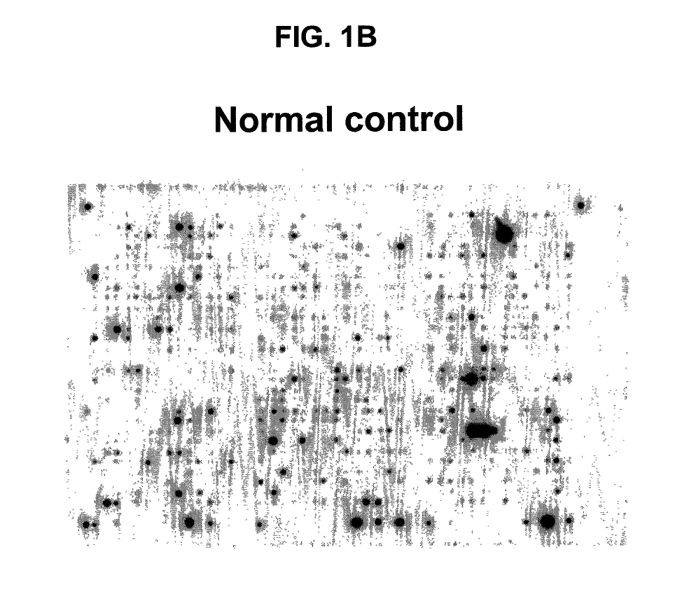

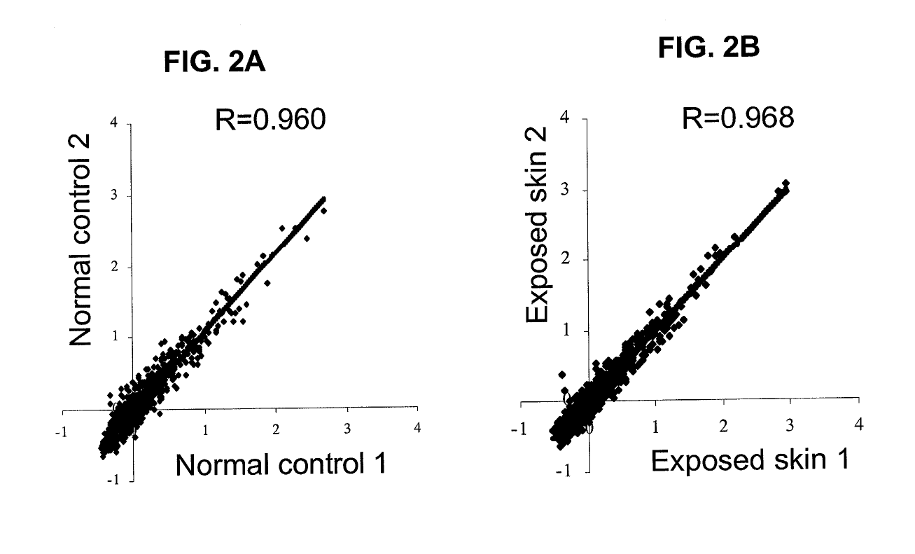

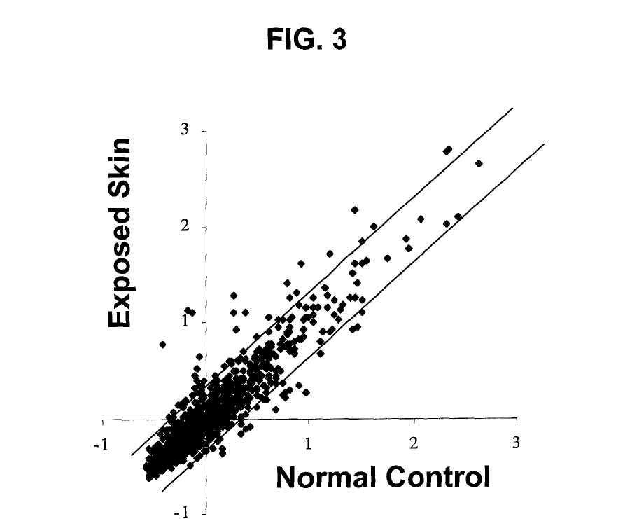

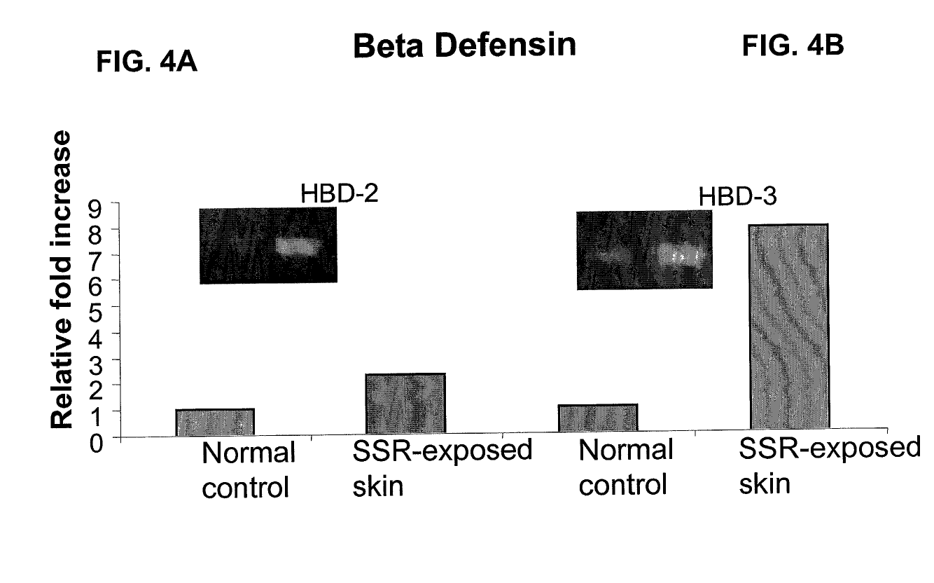

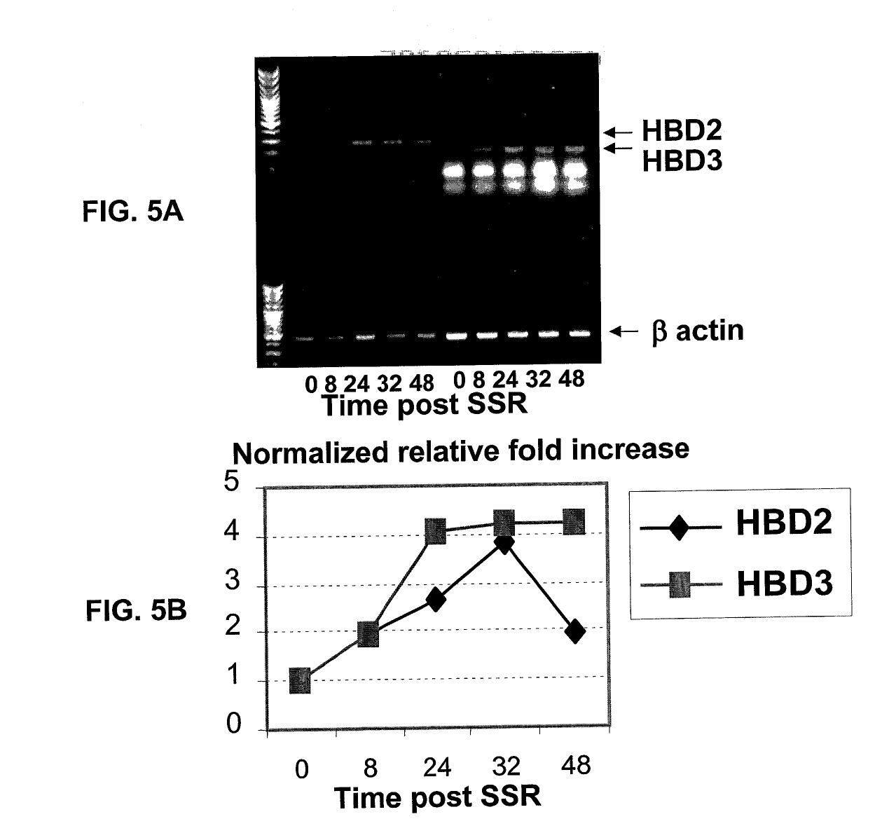

### DESCRIPTION OF THE INVENTION

Because of the harmful consequences of UV radiation-induced damage to skin, for example, immunosuppression, photocarcinogenesis, (e.g., melanoma), and wrinkles and loss of elasticity due to photoaging, there is a need to prevent, inhibit, block, reduce, treat, ameliorate, repair, and/or reverse these consequences prior to, as well as following, exposure to UV radiation.

The present invention, in general, provides a unique gene expression/regulation profile of human skin, or skin cells, under in vivo solar stimulated radiation (SSR), i.e., UV radiation, challenge. This invention has identified a new and unexpected set of genes, i.e., an array of marker genes, whose expression is altered following UV radiation exposure. The newly-discovered gene marker set includes genes comprising several different classes of protein molecules, namely, cell signaling and extra-cellular communication proteins; cell surface antigens and adhesion receptor proteins; growth factors, cytokines, chemokines and receptors; intracellular transducers, effectors and modulators; oncogenes and tumor suppressors; protein Inhibitors and protein modification molecules; xenobiotic metabolism and transporter proteins; transcriptional activators and repressor proteins; basic transcription factors; cell cycle regulators; extracellular communication proteins and transporter; and kinase activators and inhibitors. The alteration in the expression of at least one of the set of marker genes is determined relative to the expression of the genes in at least one control, such as skin, a skin substitute or a skin equivalent, that has not been exposed to UV radiation.

Specifically, the gene marker set comprises thirty-five genes of the above types whose expression is affected by UV radiation exposure. The thirty-five genes (and GenBank Accession numbers as supplied by Clontech Laboratories, Palo Alto, Calif. for the commercially available Clontech microarray) include the following: Ras related protein RAB-7 (GenBank Accession No. X93499); Corneodesmosin (GenBank Accession No. L20814); Amphiregulin (GenBank Accession No. M30704); Granulocyte Chemotatic Protein (GenBank Accession No. X78686); Migration inhibitory factor MRP8 (calgranulin A) (GenBank Accession No. X06233); Migration inhibitory factor MRP14 (calgranulin B) (GenBank Accession No. X06234); Ephrin receptor (GenBank Accession No. M59371); epithelial cell kinase (ECK) (GenBank Accession No. X74979); shb proto-oncogene (GenBank Accession No. X75342); MAD transcriptional repressor (GenBank Accession No. L06895); Calpain (GenBank Accession No. M23254); Leukocyte elastase inhibitor (monocyte/neutrophil elastase inhibitor) (GenBank Accession No. M93056); Placental plasminogen activator inhibitor (PAI-2) (GenBank Accession No. M18082; J02685); Beta-defensin (human beta defensin 2, HBD2 and human beta defensin 3, HBD3 (GenBank Accession No. Z71389), (HBD3 Accession No. M18661); Alpha 1 antitrypsin precursor (GenBank Accession No. X02920); Tristetraproline (growth factor inducible nuclear protein 475), (GenBank Accession No. M92843); Interferon regulatory factors (IFR family) (GenBank Accession No. U73036); Nuclear Factor 1 (GenBank Accession No. L31881); hSNF2 Transcriptional activator (GenBank Accession No. D26155); Prothymosin (GenBank Accession No. M26708); GATA3 transcription factor (GenBank Accession No. X55122); Histidine decarboxylase (GenBank Accession No. X54297); Acyl Co-A binding protein (GenBank Accession No. M14200); Decorin (GenBank Accession No. M14219); CD44 antigen (GenBank Accession No. M59040); B94 protein (GenBank Accession No. M92357); Transthyretin (TTR), (prealbumin) (GenBank Accession No. K02091); Apolipoprotein E (GenBank Accession No. M12529); Epithelial discoidin receptor (GenBank Accession No. X74979); Thrombin receptor (GenBank Accession No. M62424); serine/threonine protein phosphatase (GenBank Accession No. XI 2646); Leukocyte antigen-related protein (LAR) (GenBank Accession No. Y00815); Cytochrome p450 IVB1 (GenBank Accession No. J02871); Thioredoxin peroxidase (GenBank Accession No. U25182); Myristylated Alanine-Rich C Kinase Substrate, MacMARCKS (MRP) (GenBank Accession No. X70326); and EB1 microtubule associated protein (GenBank Accession No. U24166).

The genes comprising the gene marker set are presented in Table 1 and are identified by general classes therein. According to the present invention, one, all, or a combination of these genes can be employed as a unique marker, or gene marker set, for UV radiation-induced skin damage or photoaging by virtue of an alteration or change in their gene expression following exposure to UV radiation. By alteration is meant a change in gene expression relative to a control that has not been exposed to UV radiation, or a control whose exposure has been blocked or attenuated, and can include, for example, up regulation or down regulation of gene expression following UV radiation exposure. An alteration in gene expression of one or more of the genes presented herein is considered to be significant relative to control if the alteration is at least about 1.5-fold, preferably about 2-fold, more preferably, about 2-fold or greater, or about 2 standard deviations (SD) or more from the mean relative to control.

TABLE 1

| | Change/Alteration in UV | |
|---|---|---|
| Gene Category; | Radiation Induced Gene | |
| Gene Markers | Expression | Fold Change |
| Cell Signaling & | | |
| Extracellular | | |
| Communication Proteins | | |
| Ras related protein RAB-7 | Up-regulated | 2.6 |
| (Accession No. X93499) | | |
| Histidine decarboxylase | Down-regulated | 2.8 |
| (Accession No. X54297) | | |
| Acyl Co-A binding protein | Down-regulated | 3.3 |
| (Accession No. M14200) | | |
| Cell Surface Antigens & | | |
| Adhesion Receptor | | |
| Corneodesmosin | Up-regulated | 2.4 |
| (Accession No. L20814) | | |
| Decorin | Down-regulated | 3.2 |
| (Accession No. M14219) | | |
| CD44 Antigen | Down-regulated | 2.5 |
| (Accession No. M59040) | | |
| Growth Factors, | | |
| Cytokines, Chemokines, | | |
| Receptors | | |
| Amphiregulin | Up-regulated | 5.0 |
| (Accession No. M30704) | | |
| Migration Inhibitory Factor | Up-regulated | 2.8 |
| MRP8 (calgranulin A) | | |
| (Accession No. X06233) | | |
| Migration Inhibitory Factor | Up-regulated | 2.6 |
| MRP14 (calgranulin B) | | |
| (Accession No. X06234) | | |
| Granulocyte Chemotactic | Up-regulated | 3.9 |
| Protein | | |
| (Accession No. X78686) | | |
| Intracellular Transducers, | | |
| Effectors, Modulators | | |
| Ephrin receptor (epithelial | Up-regulated | 3.5 |
| cell kinase (ECK)) | | |
| (Accession No. M59371) | | |
| Epithelial discoidin | Down-regulated | 2.6 |
| receptor | | |
| (Accession No. X74979) | | |
| Thrombin receptor | Down-regulated | 3.2 |
| (Accession No. M62424) | | |
| Oncogene & Tumor | | |
| Suppressors | | |
| shb proto-oncogene | Up-regulated | 2.3 |
| (Accession No. X75342) | | |
| MAD protein | Up-regulated | 2.4 |
| (Accession No. L06895) | | |
| EB1 protein | Up-regulated | 2.3 |
| (Accession No. U24166) | | |
| Protein Inhibitors & | | |
| Protein Modification | | |
| Calpain | Up-regulated | 2.8 |
| (Accession No. M23254) | | |
| Leukocyte elastase | Up-regulated | 6.9 |
| inhibitor | | |
| (monocyte/neutrophil | | |
| elastase inhibitor) | | |
| (Accession No. M93056) | | |
| Placental plasminogen | Up-regulated | 5.1 |
| activator inhibitor (PAI-2) | | |
| (Accession Nos. M18082, | | |
| J02685) | | |
| Alpha 1 anti-trypsin | Up-regulated | 2.3 |
| precursor | | |
| (Accession No. X02920) | | |
| serine/threonine protein | Down-regulated | 2.4 |
| phosphatase | | |
| (Accession No. X12646) | | |
| Leukocyte antigen-related | Down-regulated | 3.3 |
| protein (LAR) | | |
| (Accession No. Y00815) | | |
| Xenobiotic Metabolism & | | |
| Transporters | | |
| Beta-defensin (HBD2; | Up-regulated | 21.4 |
| HBD3) | | |
| (Accession No. Z71389) | | |
| Cytochrome p450 IVB1 | Down-regulated | 3.5 |
| (Accession No. J02871) | | |
| Thioredoxin peroxidase | Down-regulated | 3.7 |
| (Accession No. U25182) | | |
| Transcriptional Activators | | |
| & Repressors | | |
| Tristetraproline (growth | Up-regulated | 2.7 |
| factor inducible nuclear | | |
| protein 475) | | |
| (Accession No. M92843) | | |
| Interferon regulatory | Up-regulated | 2.3 |
| factors (IRF family) | | |
| (Accession No. U73036) | | |
| Basic Transcription | | |
| Factors | | |
| Nuclear Factor 1 | Down-regulated | 2.3 |
| (Accession No. L31881) | | |
| hSNF2 Transcriptional | Down-regulated | 2.5 |
| Activator | | |
| (Accession No. D26155) | | |
| Cell Cycle Regulators | | |
| Prothymosin | Down-regulated | 2.5 |
| (Accession No. M26708) | | |
| GATA3 transcription | Down-regulated | 3.6 |
| factor | | |
| (Accession No. X55122) | | |
| Extracellular | | |
| Communication Proteins & | | |
| Transporters | | |
| B94 protein | Down-regulated | 2.3 |
| (Accession No. M92357) | | |
| Transthyretin (TTR), | Down-regulated | 3.2 |
| (prealbumin) | | |
| (Accession No. K02091) | | |
| Apolipoprotein E | Down-regulated | 5.0 |
| (Accession No. M12529) | | |
| Kinase Activators & | | |
| Inhibitors | | |
| MacMARCKS (MRP) | Up-regulated | 2.1 |
| (Accession No. X70326) | | |

One embodiment of the present invention embraces a method of evaluating damage to skin from UV radiation. The method comprises irradiating skin, organotypic skin models, including human and non-human animal skin, skin equivalents, human or non-human cultured cells, or keratin forming cells (e.g., those that form skin, hair and nails) with ultraviolet radiation, isolating nucleic acid, preferably RNA, by methods known and practiced in the art, and evaluating the isolated nucleic acid, preferably RNA, to determine if there are changes or alterations in the expression levels of one or more of the genes as set forth in Table 1. (See, e.g., Example 1).

According to the present method, skin (e.g., a skin biopsy or a skin keratome), an organotypic skin model, or cultured cells, is/are exposed to UV radiation or a UV radiation source for a given time period. The amount of time of UV radiation exposure is determined by the practitioner according to the MED value of an individual undergoing exposure. In general, the typical UV exposure for cells and tissue equivalents is less than or about 6 J/cm². In one of its aspects, the use of an in vivo skin source advantageously provides a sample comprising those components of skin that contribute to UV radiation induced gene expression in skin, such as vasculature, and the full complement of dermal cells and inflammatory cells that migrate into the skin in vivo.

Nonlimiting examples of cultured skin cells suitable for use in the method include primary isolation cells and established cell lines for epidermal and dermal cell types, such as, but not limited to, keratinocytes, dermal fibroblasts, Langerhans' cells, melanocytes, mast cells, endothelial cells, sebocytes, hair papilla and matrix cells, and nail matrix cells. Sources of such cells include, but are not limited to, the American Type Culture Collection (ATCC), Manassas, Va.; and from cell/tissue banks, such as Clonetics/BioWhittaker (San Diego, Calif.) or Cascade Biologics, Inc. (Portland, Oreg.). Tissue equivalents can be obtained from tissue engineering companies, such as Organogenesis, Inc. (Canton, Mass.), MatTek Corporation (Ashland, Mass.) and Skinethic, Inc. (Nice, France). Fresh cells can be obtained from biopsies of human skin or other mammals. Other skin cell lines can also be obtained from other mammals from the above sources.

Gene expression can be evaluated at one or more times following UV radiation exposure using a variety of molecular procedures for analyzing nucleic acids or polynucleotides, e.g., DNA, RNA, cDNA, isolated from the skin, skin substitute, skin equivalent, keratin forming cells, or cultured cells, for example. As a guide, evaluation of the effect of UV radiation on one or more of the gene markers can be carried out from minutes to hours to several days after UV radiation exposure, including, but not limited to, from about 5 minutes to about 96 hours, preferably from about 1 hour to about 72 hours, and more preferably from about 4 hours to about 32 hours.

Following UV radiation exposure of skin, a skin substitute, or cultured cells, the UV radiation induced alteration of the expression of one, or more than one (i.e., a combination), of the newly provided gene markers relative to one or more controls (not exposed to UV radiation) as described herein, is measurable by a number of suitable molecular techniques and procedures commonly practiced by those having skill in the art. For example, gene expression can be measured by the determination of RNA levels of the UV exposed skin, skin substitute, or cultured cells, using techniques such as Northern blot technology and PCR, e.g., “real time” PCR and reverse transcription PCR, RT PCR, as practiced in the art. (see, e.g., J. Sambrook et al., 1989, Molecular Cloning: A Laboratory Manual, Cold Spring Harbor Laboratory, Cold Spring Harbor, N.Y.; R. Higuchi et al., 1992, Biotechnology, 10:413-417; R. Higuchi et al., 1993, Biotechnology, 11:1026-1030; E. S. Kawasaki, 1990, “Amplification of RNA”, In: RNA Protocols: A Guide to Methods & Applications, M. A. Innis et al., Academic Press, San Diego, Calif., pp. 21-27). In addition, gene expression in skin, skin substitute, or cultured cells can be evaluated using gene (cDNA) arrays (microarrays or nucleic acid genechip test arrays comprising membrane, glass, or plastic support materials), serial analysis of gene expression (SAGE), (e.g., as described by V. E. Velculescu et al., 1995, Science, 270(5235):484-487; A. Lal et al., 1999, Cancer Res., 59(21):5403-5407), or differential display techniques.

Preferred is a gene array methodology, which can be readily and reliably employed in the screening and evaluation methods according to this invention. A number of gene arrays (microarrays or gene chip arrays) are commercially available for use by the practitioner, for example, but not limited to, Clontech Laboratories (Palo Alto, Calif.); Affymetrix (Santa Clara, Calif.); Operon Technologies (Alameda, Calif.); Perkin-Elmer/NEN (Boston, Mass.); and Sigma-Genosys (The Woodlands, Tex.). More specifically, nucleic acid microarray analysis allows the establishment of a pattern of gene expression from multiple genes and facilitates an understanding of the complex interactions that are elicited in a subject or a sample by selective interventions, such as exposure to UV radiation according to the present invention. The microarray procedure allows a determination of selective profile modifications among a set of genes, as well as novel and specific changes in gene expression or activity following in vivo intervention, such as UV radiation exposure. For further guidance, and without limitation, a microarray can be prepared and used according to the methods described in WO 95/11995 (Chee et al.); D. J. Lockhart et al., 1996, Nature Biotechnology, 14:1675-1680; and M. Schena et al., 1996, Proc. Natl. Acad. Sci. USA, 93:10614-10619). Microarrays are further described in the disclosure of U.S. Pat. No. 6,015,702 to P. Lal et al. In addition and as will be appreciated by those having skill in the art, high throughput analysis is encompassed as a means of analyzing the results of the microarray and/or gene chip technologies.

Prior to the present invention, the use of a gene array technique for evaluating and assessing UV radiation exposure or to identify materials that modulate gene expression resulting from exposure to UV radiation, for use in treating, ameliorating, preventing, reducing, repairing, or reversing photodamage or photoaging effects on skin was not recognized in the art. Moreover, the novel genes identified as being altered in their expression after UV exposure according to the present invention, see Table 1, had not been previously associated with photodamage or photoaging following UV radiation exposure of skin.

In evaluating the alteration in gene expression in the method according to the present invention, a difference in gene expression levels between control(s) (e.g., no UV radiation exposure, or blocked UV radiation exposure, and test sample(s) (e.g., after exposure to UV radiation) is determined. As mentioned hereinabove, in accordance with the method of the present invention, a significant difference in gene expression, i.e., that which constitutes a significant alteration or change from the control, is a difference of at least about 1.5-fold, preferably about 2-fold, more preferably, about 2-fold or greater. Alternatively in the method, a statistical difference of about two standard deviations or more from the mean is considered significant upon comparing control and test samples.

In another embodiment encompassed by the present invention, the method of evaluating gene expression alterations of one or more of the gene markers provided herein is employed to identify and/or assess ingredients, components, agents, materials, and the like, which modulate, preferably, attenuate, UV radiation induced photodamage or photoaging of skin. In this aspect, particular ingredients of skin care and/or cosmetic products can be assessed to determine their ability to modulate an alteration of expression of one or more of the gene markers described herein in skin, a skin substitute, skin cells, or keratin forming cells that has/have been exposed to UV radiation. Such a modulation by the ingredient or product can result in the prevention inhibition, blocking, reduction, treatment, amelioration, and/or reversing of photodamaged and photoaged skin.

More specifically, an ingredient, component, material, or substance undergoing testing, or a product, such as a sunscreen formulation, or an anti-photoaging formulation or composition, is contacted with, or applied to, the skin or skin substitute or an area thereof in an effective concentration and for an effective period of time, e.g., daily, weekly, or every two to four weeks. In addition, multiple applications can occur within those time periods, if needed or desired. As a nonlimiting guide, the concentration of a test material applied to a site on the skin for testing is about 0.5-5 mg/cm², preferably about 1-2 mg/cm², preferably daily, for about 1, 2, or 4 weeks.

In the evaluation method aspect of this invention, the test skin is exposed to a source of UV radiation, e.g., a minimal erythemal dose (MED), from about 0.5 MED to about 5 MED, preferably about 1 MED to about 4 MED for from about 5 minutes to about 48 hours, preferably from about 5 minutes to 1 hour. As a control, gene expression levels of one or more of the new gene marker set are examined from another skin sample that has not been exposed to UV radiation. Skin or a skin substitute without the applied test material can be exposed to UV radiation and also assessed as a control, i.e., skin or skin substitute, in the absence of test material is also exposed to UV radiation as a control.

In a preferred microarray system according to the present invention, RNA is prepared and isolated from the UV-radiation exposed skin, with and without applied test material, as well as from skin sample that did not undergo UV radiation exposure. The RNA is reverse transcribed to create a complementary nucleotide sequence (cDNA) with specific gene array primers, while incorporating radioactive ³²P label, to serve as probes for the array, for example. Labeled cDNA is then hybridized to a human gene array (e.g., a membrane substrate) comprising hundreds or thousands of genes, including the thirty-five genes described herein (e.g., Example 1).

Alternatively and preferably, the labeled cDNA is hybridized to an array (e.g., nylon or nitrocellulose membrane, or plastic film, or gene chip) containing at least one, a combination of, or all of the thirty-five genes, or nucleic acid portions (e.g., oligomers) thereof that specifically or uniquely identify the marker gene(s) newly described by the present invention, for more specific expression analysis of this unique panel of marker genes. Thus, at least one, or all, or a combination of the gene marker set as described herein can be used to prepare a specialized microarray. Preferably, a marker gene set comprising one or more, or all, of a set of about thirty-five genes, or specific subsequences thereof, is used in an array, e.g., gene (nucleic acid) chip or microarray for, e.g., isotopic blot hybridization. Nonlimiting examples of supports for arrays include nylon or nitrocellulose membrane, glass (e.g., glass slide for fluorescence detection) or plastic film support-based microarray for use in the method for determining if a product, ingredient, or material is photoprotective, or can ameliorate, prevent, reduce, repair, inhibit, block, attenuate, suppress, treat, or reverse photodamage and/or photoaging associated with UV radiation exposure, preferably of skin. That is, the method allows a determination of whether a product, ingredient, or material modulates the expression of, or an alteration in the expression of, one or more of the genes in the marker gene set following exposure to UV radiation, relative to control. It will be appreciated by the skilled practitioner that nucleic acid (e.g., cDNA isolated and prepared from skin or a skin substitute) can be labeled, or labeled probes can be suitably employed, using radioactive (e.g., ³²P) or non-radioactive materials, for example, fluorescent label, chemiluminescent label, enzyme label, biotin-avidin label, and the like, in the microarray procedures. The various labeled probes are exposed to the gene microarray. The amount of expression is demonstrated by the amount of signal (e.g., radioactive, fluorescent, chemiluminescent, etc. signal) that is obtained from the labeled probe at the specific location for that gene on the microarray. The greater the amount of binding of a probe to a specific gene location on the microarray, the greater the expression of that gene.

After an appropriate hybridization interval (e.g., from several hours to overnight or somewhat longer), the array (e.g., membrane) is washed to remove unbound material, (e.g., according to membrane supplier instructions) and placed on a phospho-imaging screen to allow visualization of gene expression levels. Gene induction is expressed as density values and is analyzed with the appropriate software (e.g., as described in Example 1) as practiced in the art. Alterations in the level of gene expression of one, or more than one, e.g., two or more, three or more, four or more, all, or a combination thereof, of the marker genes whose expression is induced by UV radiation exposure, and whose expression is altered compared with control gene expression levels, are assessed using labeled probes and detection procedures of the microarray analysis as described herein (see, e.g., Example 1).

If the test material is found to modulate an alteration or change in gene expression of one or more of the genes among the novel marker gene set of the invention after UV radiation exposure of skin, relative to the control not exposed to UV radiation, then that material is determined to be a candidate for ameliorating, preventing, reducing, inhibiting, blocking, suppressing, repairing, reversing, or treating photodamaged and/or photoaged skin. Time course experiments can also be conducted to determine the effect of the test material as a photodamage and/or photoaging protectant, or as a photodamage and/or photoaging repair or treatment agent, over prolonged or different times of exposure to UV light. Also contemplated by the present invention is a method for assessing whether a substance can reverse photodamage and/or photoaging of skin caused by UV radiation exposure by applying the described method after photodamage or photoaging has occurred.

If an ingredient, substance, material, or product undergoing testing is found to affect or modulate gene expression of one or more genes of the set of marker genes described herein in skin or test material that has been exposed to UV radiation, then the ingredient can be considered as a candidate for use in a skin care product or cosmetic formulation for topical and/or oral use to prevent, inhibit, block, reduce, treat, ameliorate, repair, or reverse photodamage and/or photoaging due to UV radiation exposure. Such skin care products and formulations for which ingredients and materials can be tested, and subsequently included, comprise, but are not limited to, sunscreens, antioxidant formulations, transdermal devices, such as patches and the like, hair care products, makeups and cosmetic products (e.g., lipsticks, facial and hand creams, foundation, body creams, lotions, moisturizers, anti-wrinkle formulations, and the like). As will be appreciated by the skilled practitioner, such materials and ingredients are preferably used in compositions and formulations in an amount effective to modulate the expression of at least one of the genes of the marker gene set, more preferably, relative to gene expression of at least one control that has not been exposed to UV radiation.

The present invention provides an advantageous and useful method to screen and evaluate the potential benefits of skincare, color, personal care, and haircare products. In view of the above description, it will be understood that the present invention further embraces a method or assay system for the identification of new materials that provide photoprotection (e.g., for prophylactic use), that repair, ameliorate, reduce, or treat photodamaged or photoaged skin, or that reverse prior photodamage (e.g., for therapeutic use) to skin that is exposed to UV radiation.

Compositions embraced by this invention can be provided in any cosmetically suitable form, preferably as a lotion or cream, but also in an ointment or oil base, as well as in a sprayable liquid form (e.g., a “hair” spray that protects hair and scalp against UV radiation induced damage, in a base that dries in a cosmetically acceptable way without the greasy appearance that a lotion or ointment would have if applied to the hair). In addition, the compositions contemplated by this invention can include one or more compatible cosmetically acceptable adjuvants commonly used and known by the skilled practitioner, such as colorants, fragrances, emollients, humectants, preservatives, vitamins, chelators, thickeners, and the like, as well as botanicals such as aloe, chamomile, and the like. If retinoids should be included, they are preferably used topically, at concentrations of between about 0.001% to about 5%, more preferably between about 0.1% to about 1%.

In another of its aspects, the present invention encompasses a method for identifying and evaluating nutritional supplements that can ultimately serve as orally ingested photoprotective, phototherapeutic and photoreparative agents. In this aspect, nutritional supplements are identified and evaluated by way of the method of the invention in which the supplement, or potential supplement, is assessed for its ability to modulate gene expression alterations of at least one of the gene markers according to the present invention after exposure to UV radiation. Such modulation can, for example, attenuate, prevent, inhibit, block, reduce, ameliorate, repair, and/or reverse UV-induced photodamage or photoaging. The present invention thus affords a new and beneficial procedure for the evaluation of the photoprotective and/or therapeutic effects of nutritional supplements, which can provide internal protection or therapy against, or ameliorate, repair, inhibit, reduce, or treat, UV-induced cell and tissue damage internally, for example. (e.g., Chakrabaty et al., 1994, Free Radical Biol. Med., 16:417).

As but one example, retinoids, e.g., retinoic acid, retinol-related materials, antioxidants, as well as the photoprotective and/or therapeutic components embraced and identified by the methods of the present invention, can also be taken systemically, preferably by oral administration. When dosed orally, retinoids and other ingested photoprotectant components are preferably administered in amounts from about 0.1 mg/kg (of body weight) to about 1 mg/kg or even more, where all doses are below that at which toxicity is likely. As another example, antioxidants are frequently taken in “megadoses” (e.g., at least 1 g/d of vitamin C, at least 1000 I.U. of one or more tocopherols).

For non-topical administration, e.g., systemic or oral administration, components or materials identified and embraced by the methods of the present invention are typically formulated in a physiologically-acceptable composition, preferably a pharmaceutically acceptable composition, including a physiologically acceptable carrier, diluent, or excipient. The compositions can be administered alone, or in combination with at least one other agent, such as a stabilizing compound, which can be administered in any sterile, biocompatible pharmaceutical carrier, including, but not limited to, saline, buffered saline, dextrose, and water. The compositions can be administered to a patient alone, or in combination with other agents, drugs, hormones, or biological response modifiers. The compositions can be ingested, administered, applied, or introduced in the form of, for example and without limitation, gel caps, tablets, powders, suspensions, liquids, caplets, bars, shakes, drinks and the like, such as further described below.

The pharmaceutical compositions for use in the present invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, vaginal, or rectal means.

Embraced by the present invention are transdermal modes of delivery, such as patches and the like, with or without a suitable permeation enhancer. The methods and compositions embodied by the invention provide a means by which one or more photoprotective, phototherapeutic, or photoreparative drugs or medicaments, can be effectively administered in a transdermal system. Frequently, compounds having poor topical absorption, or which are required at high dosage levels, are delivered transdermally. Accordingly, a transdermal means of delivering a drug composition (often with a permeation enhancing composition) to the skin is that of the transdermal patch or a similar device as known and described in the art. Examples of such devices are disclosed in U.S. Pat. Nos. 5,146,846, 5,223,262, 4,820,724, 4,379,454 and 4,956,171; such descriptions are not meant to be limiting. The transdermal mode of storing and delivering the compositions onto the skin and forming the active composition is convenient and well suited for the purposes of an embodiment of the present invention.

In addition to the active ingredients, including the photoprotective, photoreparative and/or phototherapeutic compounds or ingredients identified as described herein, the physiologically acceptable and pharmaceutical compositions can contain suitable pharmaceutically acceptable carriers, diluents, or excipients comprising auxiliaries which facilitate processing of the active compounds into preparations which can be used pharmaceutically. Further details on techniques for formulation and administration are provided in the latest edition of Remington's Pharmaceutical Sciences (Mack Publishing Co.; Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

Pharmaceutical preparations for oral use can be obtained by the combination of active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents can be added, such as cross-linked polyvinyl pyrrolidone, agar, alginic acid, or a physiologically acceptable salt thereof, such as sodium alginate.

Dragee cores can be used in conjunction with physiologically suitable coatings, such as concentrated sugar solutions, which can also contain gum arabic, talc, polyvinylpyrrolidone, carbopol gel, polyethylene glycol, and/or titanium dioxide, lacquer solutions, and suitable organic solvents or solvent mixtures. Dyestuffs or pigments can be added to the tablets or dragee coatings for product identification, or to characterize the quantity of active compound, i.e., dosage.

Pharmaceutical preparations, which can be used orally, include push-fit capsules made of gelatin, as well as soft, scaled capsules made of gelatin and a coating, such as glycerol or sorbitol. Push-fit capsules can contain active ingredients mixed with a filler or binders, such as lactose or starches, lubricants, such as talc or magnesium stearate and, optionally, stabilizers. In soft capsules, the active compounds can be dissolved or suspended in suitable liquids, such as fatty oils, liquid, or liquid polyethylene glycol with or without stabilizers.

Pharmaceutical formulations suitable for parenteral administration can be formulated in aqueous solutions, preferably in physiologically compatible buffers such as Hanks' solution, Ringer's solution, or physiologically buffered saline. Aqueous injection suspensions can contain substances which increase the viscosity of the suspension, such as sodium carboxymethyl cellulose, sorbitol, or dextran. In addition, suspensions of the active compounds can be prepared as appropriate oily injection suspensions. Suitable lipophilic solvents or vehicles include fatty oils such as sesame oil, or synthetic fatty acid esters such as ethyloleate or triglycerides, or liposomes. Optionally, the suspension can also contain suitable stabilizers or agents which increase the solubility of the compounds to allow for the preparation of highly concentrated solutions. As will be appreciated by the person having skill in the art, the preparation of pharmaceutical formulations in their various forms will not adversely affect the function or activity of the active materials in the formulation.

For topical or nasal administration, penetrants or permeation agents or enhancers that are appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes.

If applicable, the pharmaceutical composition can be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preparation can be a lyophilized powder which can contain any or all of the following: 1-50 mM histidine, 0.1%-2% sucrose and 2-7% mannitol, at a pH range of 4.5 to 5.5, combined with a buffer prior to use.

After the photoprotective, photoreparative and/or phototherapeutic pharmaceutical compositions have been prepared, they can be placed in an appropriate container and labeled for treatment of an indicated condition. For administration of the photoprotective, photoreparative and/or phototherapeutic product, such labeling would include amount, frequency, and method of administration.

Pharmaceutical compositions suitable for use in the present invention include compositions in which the active ingredients or materials are contained in an amount effective to achieve the intended purpose. The determination of an effective dose or amount is well within the capability of those skilled in the art. For any compound, the therapeutically effective dose or concentration range can be estimated initially in cell culture assays, e.g., using neoplastic cells. A therapeutically effective dose refers to that amount of active ingredient, for example, a photoprotective, photoreparative and/or phototherapeutic compound or component identified in accordance with the present invention, which, for instance, prevents, ameliorates, reduces, treats, reverses, suppresses, repairs, or eliminates the symptoms or condition. Dosage generally varies within this range depending upon the dosage form employed, sensitivity of the patient, and the route of administration. The practitioner, who will consider the factors related to the individual requiring treatment, will determine the exact dosage.

Dosage and administration are adjusted to provide sufficient levels of the active moiety or to maintain the desired effect. Factors which are typically considered include the severity of the individual's particular need, general health of the patient, age, weight, and gender of the patient, diet, time and frequency of administration, drug combination(s), reaction sensitivities, and tolerance/response to treatment. As a general guide, long-acting pharmaceutical compositions can be administered every 3 to 4 days, every week, or once every two weeks, depending on half-life and clearance rate of the particular formulation. Variations in these dosage levels can be adjusted using standard empirical routines for optimization, as is well understood in the art.

As a nonlimiting guide, normal dosage amounts can vary from 0.1 to 100,000 micrograms (μg), up to a total dose of about 1 gram (g), depending upon the route of administration. Guidance as to particular dosages and methods of delivery is provided in the literature and is generally available to practitioners in the art. Those skilled in the art will employ different formulations depending upon the nature, e.g., structure, composition, of the photoprotective and/or phototherapeutic compound.

In another embodiment, the present invention provides a screening method to allow the identification of individuals, or a subset of individuals, who respond to UV induced gene expression by having altered expression of one or more of the marker genes in the UV radiation-induced marker gene set as newly-described herein. Such individuals, who are likely to be more susceptible or highly sensitive than normal to skin photodamage and photoaging following exposure to UV radiation as a result of their having an alteration in the expression of one or more of the UV radiation induced marker genes, are able to be identified through screening using the methods of the present invention. For example, a screening method of this embodiment involves obtaining from an individual undergoing testing a skin sample, such as a skin biopsy or skin keratome sample, e.g., as described in Example 1; exposing the sample to UV radiation; isolating nucleic acid from the UV radiation exposed sample; and employing an assay method, e.g., the microarray system and newly-identified marker genes as described, to evaluate whether gene expression of at least one of the UV radiation induced genes in the individual is altered relative to control. The screening method further allows the determination of those individuals who are particularly amenable to treatment or therapies involving materials, ingredients, compounds, formulations and compositions that are capable of modulating, or effecting modification of, an alteration in the expression of one or more genes of the UV radiation induced marker gene set as described herein. The identified or screened individuals would be particularly suited to amelioration, reduction, treating, preventing, repairing, reducing, and or reversing of UV radiation induced photodamage and photoaging according to the present invention.

Yet another embodiment of the present invention relates to a kit containing a support or support material, such as, without limitation, a nylon or nitrocellulose membrane, or plastic film, or glass, or microarray, comprising the novel set of genes as described herein, in a form suitable for the practitioner to employ in identifying compounds, reagents, ingredients, substances, agents, and the like, which can ameliorate, prevent, inhibit, block, suppress, reduce, repair, treat or reverse photodamage and/or photoaging caused by UV radiation exposure of skin. The kit can contain the novel gene marker set, or a subset of these genes, or unique nucleic acid portions of these genes, on the suitable substrate or microarray, which provides an identified target set of gene markers for identifying agents that are able to modulate (i.e., attenuate, effect modification, or overcome) UV radiation-induced photodamage and/or photoaging of skin. In addition, the kit can optionally contain other materials necessary for carrying out the assay method, including but not limited to, labeled or unlabeled nucleic acid probes, detection label, buffers, controls, and instructions for use.

### EXAMPLES

The following examples describe specific aspects of the invention to illustrate the invention and provide a description of the present methods for those of skill in the art. The examples should not be construed as limiting the invention, as the examples merely provide specific methodology useful in understanding and practice of the invention and its various aspects.

### Example 1

Cells respond to stimuli via genomic and non-genomic changes in cellular function. Understanding how a cell and tissue respond at the genomic (RNA) level to a given stimulus has traditionally been conducted on an individual (single) gene basis. With the utilization of gene array techniques, several hundred, and even thousands, of genes can be studied in a single experiment.

The purpose of the experiment described in this example was to utilize the gene array technology and human skin so as to identify changes that result in response to photodamage caused by ultraviolet radiation (UVR). Because UVR is the etiologic agent responsible for photodamage and photoaging of skin, UV irradiated skin was used as a model system to examine acute and chronic changes associated with photodamage. This model system, based on human skin samples, was used as a method to screen materials that can ameliorate, as well as reduce, treat, repair, reverse, or prevent, the effects of photodamage caused by both acute and chronic UVR exposures.

To perform the experiments, skin keratomes, approximately 2 cm×5 cm, containing epidermal and dermal cells were collected from the buttock region of 5 normal volunteers. Keratomes were collected at 32 hours after a 4 MED UVR exposure (solar simulated radiation, SSR). Control keratomes received no UVR exposure. Tissues were snap frozen for subsequent total RNA isolation.

For RNA isolation, tissues were homogenized by polytron in Trizol reagent (Invitrogen Life Technologies, Carlsbad, Calif.), pooled, and total RNA was isolated per the Trizol reagent instructions. Once isolated, total RNA was reverse transcribed to create a complementary nucleotide sequence (cDNA) with a specific gene array primers set (Clontech Laboratories, Palo Alto, Calif.) while incorporating radioactive ³²P label. Labeled-cDNA was hybridized to a human Atlas 1.2 I Array containing 1176 human genes (Clontech Laboratories, Palo Alto, Calif.). After an overnight hybridization, membranes were washed according to membrane supplier instructions and placed onto phospho-imaging screens for approximately 10 days to allow visualization of gene expression levels. Gene induction, expressed as density values, was obtained and analyzed with Atlasimage software (Clontech Laboratories, Palo Alto, Calif.) via the phosphoimager analyzer. After gene expression was quantified by phosphoimage analysis, expression of genes was normalized to the average density of all of the genes on the membrane and corrected for background hybridization using ImageQuaNT™ software.

The entire RNA extraction, labeling, hybridization, and calculation of the gene changes were repeated four times and averaged. Gene expression changes of about 2 to 2.5-fold relative to control, or two standard deviations (SD), either above or below baseline (control), were considered significant in this example. Those genes which showed significant changes in expression following UV radiation exposure of the skin tissue, either upregulation or downregulation, are described herein and are presented in Table 1.

### Example 2

### Microarrays

For the production of oligonucleotides specific for the genes in a microarray, specific gene sequence(s) is/are examined using a computer algorithm which starts at the 3′ end of the nucleotide sequence. The algorithm identifies oligomers of defined length that are unique to the gene, have a GC content within a range that is suitable for hybridization and lack predicted secondary structure that would interfere with hybridization. The algorithm identifies specific oligonucleotides of particular length, e.g., 20 to 100 nucleotides, e.g., 20-mers, 30-mers, 50-mers, 80-mers, 100-mers. A matched set of oligonucleotides is created in which one nucleotide in the center of each sequence is altered. This process is repeated for each gene in the microarray, and double sets of oligos are synthesized in the presence of fluorescent or radioactive nucleotides and arranged on the surface of a substrate. When the substrate is a silicon chip, a light-directed chemical process is used for deposition (see, e.g., WO 95/11995, M. Chee et al.).

Alternatively, a chemical coupling procedure and an ink jet device is used to synthesize oligomers on the surface of a substrate. (see, e.g., WO 95/25116, J. D. Baldeschweiler et al.). As another alternative, a “gridded” array that is analogous to a dot (or slot) blot is used to arrange and link cDNA fragments or oligonucleotides to the surface of a substrate using, for example, a vacuum system, or thermal, UV, mechanical, or chemical bonding techniques.

A typical array may be produced by hand, or by using available materials and equipment, and may contain grids of multiple dots. After hybridization, the microarray is washed to remove any non-hybridized probe, and a detection device is used to determine the levels and patterns of radioactivity or fluorescence. The detection device may be as simple as X-ray film, or as complicated as a light scanning apparatus. Scanned fluorescent images are examined to determine degree of complementarity and the relative abundance/expression level, or changes in expression levels, of each oligonucleotide sequence in the microarray, preferably by comparison of a control and a test sample or material.

### Example 3

### Utilization of Two Types of Gene Array Methodologies for In Vitro Tissue Models or Monolayer Cultures

In vitro human epidermis tissue (size 0.63 cm²) or monolayer cultures plated in 100 mm tissue culture dishes (70-80% confluence) are fed with fresh medium 24 hours prior to test exposure. The test ingredient samples are diluted in the medium or applied topically for tissue to the models 1 hour prior to UV exposure.

For monolayer culture exposures, all available cell culture medium is decanted from the dish immediately (e.g., about 5 minutes) prior to UV exposure. In tissues, any excess material is removed from the topical application by a saline (physiological) wash to avoid direct interaction with the UV light. After UV radiation exposure, three tissues/monolayer cultures are used for each treatment, including an untreated control (contains only medium), a UV treated (UV control) and an untreated/non-UV control (baseline). Tissues are stored frozen at −80° C. until RNA isolation.

Total RNA is isolated from the cell cultures or tissue (tissue models) using the Clontech Nucleospin RNA II Purification kit (Clontech Laboratories, Inc., Palo Alto, Calif.). The tissue models are homogenized using a commercial homogenizer or are ground with a mortar and pestle in the presence of liquid nitrogen. The homogenized tissue models or monolayer cell cultures are lysed with lysis buffer supplied by the supplier of the kit, and the resulting lysed material is then pipetted into a homogenous suspension. (If the cell suspension is turbid due to the presence of some remaining intact cells or particles, or is very viscous from genomic DNA, the samples are filtered using a Nucleospin filter unit (Clontech Laboratories, Inc., Palo Alto, Calif.)). A prepared DNase I Reaction mixture is added to the Nucleospin column and incubated for 15 minutes at room temperature. After several washings with buffer as supplied, the Nucleospin filter tube is placed into a 1.5-ml tube at which time the RNA is eluted using Nuclease free water. RNA purity and yield are measured using UV spectroscopy and the absorbency ratio of 260 nm/280 nm is determined. The RNA is also checked for purity and integrity using denaturing agarose gel electrophoresis as is commonly known in the art.

For each endpoint measured, 10-20 μg of total RNA is utilized for hybridization. Total RNA is labeled using either the Clontech Atlas Fluorescent Labeling Kit (Clontech Laboratories) and Cy3 and Cy5 labels (Amersham Pharmacia, Piscataway, N.J.), or is radiolabeled with ³³P or ³²P dATP (Amersham Pharmacia) using the Atlas cDNA expression Array Kit (Clontech Laboratories) and following the kit instructions provided by the manufacturer.

The labeled RNA is applied onto a glass slide (fluorescent-labeled RNA) or onto a nylon membrane (radiolabeled RNA) for hybridization by diluting it with corresponding amount of hybridization solution as supplied in the kit so as to cover the glass slide or nylon membrane. After the incubation (approximately 16 hours), the glass slide or membrane is washed with various concentrations of SSC buffers, according to the manufacturer's instructions. For CY3/CY5 labeled glass slides, the slides are dried using dry nitrogen and placed in a fluorescent gene array reader (Axon Instruments, Foster Calif., Genepix 4000B), which contains lasers for exciting/emitting both Cy3 and Cy5 fluorescent markers.

For radiolabeled nylon membranes, the membranes are exposed to a phosphorimaging screen for about 6-14 days before visualization by Phosphoimager (Molecular Dynamics Inc, Sunnyvale, Calif., STORM 840).

Array analysis is conducted on fluorescent labeled glass slides using Genepix Pro 3.0 software provided with the Genepix 4000B scanner (Axon Instruments), or using AtlasImage 1.5 (Clontech Laboratories) for radiolabeled nylon membranes, followed by additional analysis using AtlasNavigator 1.0 software (Clontech Laboratories). For example, Genepix Pro 3.0 allows the quantitative analysis and comparison of signal intensities from glass slide microarrays; AtlasImage 1.5 allows the quantitative analysis and comparison of signal intensities from Atlas nylon arrays; Atlas Navigator is employed for gene cluster analysis, comprehensive normalization of array data, online gene annotations, presentations of genes by line graphs, bar graphs, scatter plots and ordered lists.

The contents of all patents, patent applications, published articles, abstracts, books, reference manuals and abstracts, GenBank sequence accession numbers, as cited herein are hereby incorporated by reference in their entireties to more fully describe the state of the art to which the invention pertains.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

What is claimed is:

1. A marker gene set comprising at least one gene identified by an alteration of expression level following exposure to ultraviolet radiation relative to the expression level following no exposure to ultraviolet radiation, said marker gene set selected from the group consisting of Ras related protein RAB-7; Corneodesmosin; Amphiregulin; Granulocyte Chemotatic Protein; Migration inhibitory factor MRP8 (calgranulin A); Migration inhibitory factor MRP14 (calgranulin B); Ephrin receptor; epithelial cell kinase (ECK); shb proto-oncogene; MAD transcriptional repressor; Calpain; Leukocyte elastase inhibitor (monocyte/neutrophil elastase inhibitor); Placental plasminogen activator inhibitor (PAI-2); Beta-defensin (human beta defensin 2, HBD2 and human beta defensin 3, HBD3); Alpha 1 antitrypsin precursor; Tristetraproline; growth factor inducible nuclear protein 475; Interferon regulatory factors (IFR family); Nuclear Factor 1; hSNF2 Transcriptional activator; Prothymosin; GATA3 transcription factor; Histidine decarboxylase; Acyl Co-A binding protein; Decorin; CD44 antigen; B94 protein; Transthyretin (TTR), (prealbumin); Apolipoprotein E; Epithelial discoidin receptor; Thrombin receptor; serine/threonine protein phosphatase; Leukocyte antigen-related protein (LAR); Cytochrome p450 IVB1; Thioredoxin peroxidase; Myristylated Alanine-Rich C Kinase Substrate, MacMARCKS (MRP); EB1 microtubule associated protein; a unique fragment thereof; and combinations thereof.

2. The marker gene set according to claim 1, wherein the alteration of expression of the at least one gene following ultraviolet radiation exposure comprises (i) at least about 1.5-fold expression level compared to control expression level, or (ii) two standard deviations or more from the mean compared to control.

3. The marker gene set according to claim 1 or claim 2, wherein the control comprises one or more of the following: (i) gene expression level of the at least one marker gene in the absence of exposure to ultraviolet radiation; or (ii) gene expression level of the at least one marker gene in the presence of a block or attenuation to ultraviolet radiation.

4. A method of evaluating the ultraviolet radiation protective, reparative, and/or therapeutic effects of a compound or material comprising:
(a) contacting skin or a skin substitute with a test compound or material undergoing evaluation;
(b) exposing the contacted skin or skin substitute to an ultraviolet radiation source; and
(c) assessing if the test compound or material modulates gene expression of at least one gene of a set of marker genes following exposure of contacted skin or skin substitute to ultraviolet radiation, compared with gene expression of a control; wherein the one or more marker genes is selected from the group consisting of Ras related protein RAB-7; Corneodesmosin; Amphiregulin; Granulocyte Chemotatic Protein; Migration inhibitory factor MRP8 (calgranulin A); Migration inhibitory factor MRP14 (calgranulin B); Ephrin receptor; epithelial cell kinase (ECK); shb proto-oncogene; MAD transcriptional repressor; Calpain; Leukocyte elastase inhibitor (monocyte/neutrophil elastase inhibitor); Placental plasminogen activator inhibitor (PAI-2); Beta-defensin (human beta defensin 2, HBD2 and human beta defensin 3, HBD3); Alpha 1 antitrypsin precursor; Tristetraproline; growth factor inducible nuclear protein 475; Interferon regulatory factors (IFR family); Nuclear Factor 1; hSNF2 Transcriptional activator; Prothymosin; GATA3 transcription factor; Histidine decarboxylase; Acyl Co-A binding protein; Decorin; CD44 antigen; B94 protein; Transthyretin (TTR), (prealbumin); Apolipoprotein E; Epithelial discoid in receptor; Thrombin receptor; serine/threonine protein phosphatase; Leukocyte antigen-related protein (LAR); Cytochrome p450 IVB1; Thioredoxin peroxidase; Myristylated Alanine-Rich C Kinase Substrate, MacMARCKS (MRP); EB1 microtubule associated protein; and combinations thereof;
wherein the ability of the test compound or material to modulate gene expression of the at least one gene of the set of marker genes relative to control indicates that the compound or material has ultraviolet radiation protective, reparative, and/or therapeutic effects.

5. The method according to claim 4, wherein the skin substitute is selected from human and non-human organotypic skin models or human and non-human cultured cells.

6. The method according to claim 4, wherein the control comprises one or more of the following: (i) the same or different skin or skin substitute that has not been exposed to ultraviolet radiation; (ii) the same or different skin or skin substitute, wherein ultraviolet radiation is blocked or attenuated; or (iii) the same or different skin or skin substitute exposed to ultraviolet radiation in the absence of the compound or material.

7. The method according to claim 4, wherein the contacting of (a) comprises topical application of the test compound or material onto the skin or skin substitute, or an area thereof.

8. The method according to claim 4, wherein the expression level of the at least one gene of the set of marker genes of the skin or skin substitute following exposure to ultraviolet radiation is selected from (i) at least about 1.5-fold compared with control expression levels, or (ii) about two standard deviations or more from the mean compared with control expression levels.

9. The method according to claim 4, wherein the skin or skin substitute is exposed to ultraviolet radiation for a period of time selected from about 5 minutes to about 96 hours; about 1 hour to about 72 hours; about 4 hours to about 32 hours, about 5 minutes to about 48 hours, or about 5 minutes to about 1 hour.

10. The method according to claim 4, wherein the skin or skin substitute is exposed to the ultraviolet radiation source at a minimal erythemal dose (MED).

11. The method according to claim 10, wherein the skin or skin substitute is exposed to the ultraviolet radiation source at a minimal erythemal dose (MED) of from about 1 MED to about 4 MED.

12. The method according to claim 4, wherein assessment is performed by an assay selected from the group consisting of microarray, Northern Blotting, polymerase chain reaction, reverse polymerase chain reaction, serial analysis of gene expression, and differential display.

13. The method according to claim 4, wherein the steps are repeated (i) over prolonged times of exposure to the ultraviolet radiation source, or (ii) for different times of exposure to the ultraviolet radiation source.

14. A compound or material having ultraviolet radiation protective, reparative, or therapeutic effects as determined by the method according to claim 4.

15. A compound or material having ultraviolet radiation protective, reparative, or therapeutic effects, wherein said compound or material modulates an alteration of gene expression of at least one gene of a set of marker genes following exposure of skin or skin substitute to ultraviolet radiation, compared with gene expression of a control; wherein the one or more marker genes is selected from the group consisting of Ras related protein RAB-7; Corneodesmosin; Amphiregulin; Granulocyte Chemotatic Protein; Migration inhibitory factor MRP8 (calgranulin A); Migration inhibitory factor MRP14 (calgranulin B); Ephrin receptor; epithelial cell kinase (ECK); shb proto-oncogene; MAD transcriptional repressor; Calpain; Leukocyte elastase inhibitor (monocyte/neutrophil elastase inhibitor); Placental plasminogen activator inhibitor (PAI-2); Beta-defensin (human beta defensin 2, HBD2 and human beta defensin 3, HBD3); Alpha 1 antitrypsin precursor; Tristetraproline; growth factor inducible nuclear protein 475; Interferon regulatory factors (IFR family); Nuclear Factor 1; hSNF2 Transcriptional activator; Prothymosin; GATA3 transcription factor; Histidine decarboxylase; Acyl Co-A binding protein; Decorin; CD44 antigen; B94 protein; Transthyretin (TTR), (prealbumin); Apolipoprotein E; Epithelial discoidin receptor; Thrombin receptor; serine/threonine protein phosphatase; Leukocyte antigen-related protein (LAR); Cytochrome p450 IVB1; Thioredoxin peroxidase; Myristylated Alanine-Rich C Kinase Substrate, MacMARCKS (MRP); EB1 microtubule associated protein; and combinations thereof;
and further wherein the ability of the test compound or material to modulate an alteration of gene expression of the at least one gene of the set of marker genes relative to control indicates that the compound or material has ultraviolet radiation protective, reparative, and/or therapeutic effects.

16. The compound or material according to claim 14 or claim 15, which is a nutritional supplement.

17. A method of ameliorating and/or treating photodamaged or photoaged skin, comprising:
applying to skin, or to an area thereof, for an effective period of time a composition containing an effective amount of a material that modulates the expression of at least one gene of a set of marker genes whose expression is altered following exposure of skin to ultraviolet radiation; said composition applied in an amount effective to modulate the expression of the at least one gene of the set of marker genes selected from the group consisting of Ras related protein RAB-7; Corneodesmosin; Amphiregulin; Granulocyte Chemotatic Protein; Migration inhibitory factor MRP8 (calgranulin A); Migration inhibitory factor MRP14 (calgranulin B); Ephrin receptor; epithelial cell kinase (ECK); shb proto-oncogene; MAD transcriptional repressor; Calpain; Leukocyte elastase inhibitor (monocyte/neutrophil elastase inhibitor); Placental plasminogen activator inhibitor (PAI-2); Beta-defensin (human beta defensin 2, HBD2 and human beta defensin 3, HBD3); Alpha 1 antitrypsin precursor; Tristetraproline; growth factor inducible nuclear protein 475; Interferon regulatory factors (IFR family); Nuclear Factor 1; hSNF2 Transcriptional activator; Prothymosin; GATA3 transcription factor; Histidine decarboxylase; Acyl Co-A binding protein; Decorin; CD44 antigen; B94 protein; Transthyretin (TTR), (prealbumin); Apolipoprotein E; Epithelial discoidin receptor; Thrombin receptor; serine/threonine protein phosphatase; Leukocyte antigen-related protein (LAR); Cytochrome p450 IVB1; Thioredoxin peroxidase; Myristylated Alanine-Rich C Kinase Substrate, MacMARCKS (MRP); EB1 microtubule associated protein; and combinations thereof.

18. A method of reversing and/or repairing photodamaged or photoaged skin, comprising:
applying to skin, or to an area thereof, for an effective period of time a composition containing an effective amount of a material that modulates the expression of at least one gene of a set of marker genes whose expression is altered following exposure of skin to ultraviolet radiation, said composition applied following ultraviolet radiation exposure in an amount effective to modulate the expression of the at least one gene of the set of marker genes selected from the group consisting of Ras related protein RAB-7; Corneodesmosin; Amphiregulin; Granulocyte Chemotatic Protein; Migration inhibitory factor MRP8 (calgranulin A); Migration inhibitory factor MRP14 (calgranulin B); Ephrin receptor; epithelial cell kinase (ECK); shb proto-oncogene; MAD transcriptional repressor; Calpain; Leukocyte elastase inhibitor (monocyte/neutrophil elastase inhibitor); Placental plasminogen activator inhibitor (PAI-2); Beta-defensin (human beta defensin 2, HBD2 and human beta defensin 3, HBD3); Alpha 1 antitrypsin precursor; Tristetraproline; growth factor inducible nuclear protein 475; Interferon regulatory factors (IFR family); Nuclear Factor 1; hSNF2 Transcriptional activator; Prothymosin; GATA3 transcription factor; Histidine decarboxylase; Acyl Co-A binding protein; Decorin; CD44 antigen; B94 protein; Transthyretin (TTR), (prealbumin); Apolipoprotein E; Epithelial discoidin receptor; Thrombin receptor; serine/threonine protein phosphatase; Leukocyte antigen-related protein (LAR); Cytochrome p450 IVB1; Thioredoxin peroxidase; Myristylated Alanine-Rich C Kinase Substrate, MacMARCKS (MRP); EB1 microtubule associated protein; and combinations thereof.

19. The method according to claim 17 or claim 18, wherein the composition is applied daily.

20. The method according to claim 17 or claim 18, wherein the composition is applied for 2-4 weeks.

21. A method of evaluating if a substance can repair or reverse photodamage or photoaging effects related to exposure to ultraviolet radiation, comprising:
(a) exposing a test material selected from skin or a skin substitute to an ultraviolet radiation source, wherein the ultraviolet radiation source exposure results in an alteration in expression level of at least one gene of a set of marker genes of the test material following exposure to ultraviolet radiation compared with control; said marker genes selected from the group consisting of Ras related protein RAB-7; Corneodesmosin; Amphiregulin; Granulocyte Chemotatic Protein; Migration inhibitory factor MRP8 (calgranulin A); Migration inhibitory factor MRP14 (calgranulin B); Ephrin receptor; epithelial cell kinase (ECK); shb proto-oncogene; MAD transcriptional repressor; Calpain; Leukocyte elastase inhibitor (monocyte/neutrophil elastase inhibitor); Placental plasminogen activator inhibitor (PAI-2); Beta-defensin (human beta defensin 2, HBD2 and human beta defensin 3, HBD3); Alpha 1 antitrypsin precursor; Tristetraproline; growth factor inducible nuclear protein 475; Interferon regulatory factors (IFR family); Nuclear Factor 1; hSNF2 Transcriptional activator; Prothymosin; GATA3 transcription factor; Histidine decarboxylase; Acyl Co-A binding protein; Decorin; CD44 antigen; B94 protein; Transthyretin (TTR), (prealbumin); Apolipoprotein E; Epithelial discoidin receptor; Thrombin receptor; serine/threonine protein phosphatase; Leukocyte antigen-related protein (LAR); Cytochrome p450 IVB1; Thioredoxin peroxidase; Myristylated Alanine-Rich C Kinase Substrate, MacMARCKS (MRP); EB1 microtubule associated protein; a unique subfragment of said marker genes; and combinations thereof;
(b) contacting the substance undergoing evaluation with the test material that has been exposed to the ultraviolet radiation source; and
(c) assessing if the substance undergoing evaluation modulates the expression level of the at least one gene of the set of marker genes of the ultraviolet radiation exposed test material so as to result in the gene expression level of the ultraviolet radiation exposed test material reflecting or attaining the gene expression level of the control; wherein the ability of the substance to modulate the expression level of the at least one gene of the set of marker genes of the ultraviolet radiation exposed test material indicates that the substance can repair or reverse the effects of ultraviolet radiation on the test material, thereby repairing or reversing the effects of skin photodamage or photoaging related to ultraviolet radiation exposure.

22. The method according to claim 21, wherein the skin substitute is selected from human and non-human organotypic skin models or human and non-human cultured cells.

23. The method according to claim 21, wherein the control comprises one or more of the following: (i) the same or different skin or skin substitute that has not been exposed to ultraviolet radiation; (ii) the same or different skin or skin substitute, wherein ultraviolet radiation is blocked or attenuated; or (iii) the same or different skin or skin substitute exposed to ultraviolet radiation in the absence of the substance.

24. The method according to claim 21, wherein the skin or skin substitute is exposed to ultraviolet radiation for a period of time selected from about 5 minutes to about 96 hours; about 1 hour to about 72 hours; about 4 hours to about 32 hours, about 5 minutes to about 48 hours, or about 5 minutes to about 1 hour.

25. The method according to claim 21, wherein the skin or skin substitute is exposed to the ultraviolet radiation source at a minimal erythemal dose (MED).

26. The method according to claim 25, wherein the skin or skin substitute is exposed to the ultraviolet radiation source at a minimal erythemal dose (MED) of from about 1 MED to about 4 MED.

27. The method according to claim 21, wherein the skin or skin substitute is exposed to the ultraviolet radiation source at a minimal erythemal dose (MED) of from about 1 MED to about 4 MED for from about 5 minutes to about 48 hours.

28. The method according to claim 21, wherein assessment is performed by an assay selected from the group consisting of microarray, Northern Blotting, polymerase chain reaction, reverse polymerase chain reaction, serial analysis of gene expression, and differential display.

29. The method according to claim 21, wherein the steps are repeated (i) over prolonged times of exposure to the ultraviolet radiation source, or (ii) for different times of exposure to the ultraviolet radiation source.

30. A substance or material having skin photodamage or photoaging reparative or reversing effects as determined by the method according to claim 21.

31. A substance or material having skin photodamage or photoaging reparative or reversing effects, wherein the substance or material modulates an alteration of gene expression level of at least one gene of a set of marker genes of skin exposed to ultraviolet radiation so as to result in the gene expression level of the ultraviolet radiation exposed skin reflecting or attaining the gene expression level of a control not exposed to ultraviolet radiation, or to which ultraviolet radiation is blocked or attenuated, thereby repairing or reversing the effects of skin photodamage or photoaging related to ultraviolet radiation exposure, said set of marker genes selected from the group consisting of Ras related protein RAB-7; Corneodesmosin; Amphiregulin; Granulocyte Chemotatic Protein; Migration inhibitory factor MRP8 (calgranulin A); Migration inhibitory factor MRP14 (calgranulin B); Ephrin receptor; epithelial cell kinase (ECK); shb proto-oncogene; MAD transcriptional repressor; Calpain; Leukocyte elastase inhibitor (monocyte/neutrophil elastase inhibitor); Placental plasminogen activator inhibitor (PAI-2); Beta-defensin (human beta defensin 2, HBD2 and human beta defensin 3, HBD3); Alpha 1 antitrypsin precursor; Tristetraproline; growth factor inducible nuclear protein 475; Interferon regulatory factors (IFR family); Nuclear Factor 1; hSNF2 Transcriptional activator; Prothymosin; GATA3 transcription factor; Histidine decarboxylase; Acyl Co-A binding protein; Decorin; CD44 antigen; B94 protein; Transthyretin (TTR), (prealbumin); Apolipoprotein E; Epithelial discoidin receptor; Thrombin receptor; serine/threonine protein phosphatase; Leukocyte antigen-related protein (LAR); Cytochrome p450 IVB1; Thioredoxin peroxidase; Myristylated Alanine-Rich C Kinase Substrate, MacMARCKS (MRP); EB1 microtubule associated protein; and combinations thereof.

32. The substance or material according to claim 30 or claim 31, which is a nutritional supplement.

33. A composition or formulation comprising the substance or material according to claim 30 or claim 31.

34. A photoprotective or therapeutic anti-photodamage or anti-photoaging formulation comprising the compound or material as determined by the method according to claim 4.

35. A method of evaluating ultraviolet radiation induced damage to skin or a skin substitute following ultra4violet radiation exposure, comprising:
(a) assessing the gene expression level of at least one gene of a set of marker genes following ultraviolet radiation exposure of the skin or skin substitute, wherein the marker genes are selected from the group consisting of Ras related protein RAB-7; Corneodesmosin; Amphiregulin; Granulocyte Chemotatic Protein; Migration inhibitory factor MRP8 (calgranulin A); Migration inhibitory factor MRP14 (calgranulin B); Ephrin receptor; epithelial cell kinase (ECK); shb proto-oncogene; MAD transcriptional repressor; Calpain; Leukocyte elastase inhibitor (monocyte/neutrophil elastase inhibitor); Placental plasminogen activator inhibitor (PAI-2); Beta-defensin (human beta defensin 2, HBD2 and human beta defensin 3, HBD3); Alpha 1 antitrypsin precursor; Tristetraproline; growth factor inducible nuclear protein 475; Interferon regulatory factors (IFR family); Nuclear Factor 1; hSNF2 Transcriptional activator; Prothymosin; GATA3 transcription factor; Histidine decarboxylase; Acyl Co-A binding protein; Decorin; CD44 antigen; B94 protein; Transthyretin (TTR), (prealbumin); Apolipoprotein E; Epithelial discoidin receptor; Thrombin receptor; serine/threonine protein phosphatase; Leukocyte antigen-related protein (LAR); Cytochrome p450 IVB1; Thioredoxin peroxidase; Myristylated Alanine-Rich C Kinase Substrate, MacMARCKS (MRP); EB1 microtubule associated protein; a unique subfragment of the marker genes; and combinations thereof; and
(b) comparing the expression level of the at least one marker gene from (a) with the expression level of a control (i) that has not been exposed to ultraviolet radiation; or (ii) wherein ultraviolet radiation to the control is blocked or attenuated, to determine if an alteration has occurred in the gene expression level of the at least one marker gene compared with the gene expression level of the control; wherein an alteration of expression level of the at least one marker gene of the marker gene set relative to control is selected from (i) at least about 1.5-fold compared to control expression levels, or (ii) about 2 standard deviations or more from the mean compared to control expression levels, and correlates with ultraviolet radiation induced damage to the skin or skin substitute.

36. The method according to claim 35, wherein the skin substitute is selected from human and non-human organotypic skin models or human and non-human cultured cells.

37. The method according to claim 35, wherein the skin or skin substitute is exposed to ultraviolet radiation for a period of time selected from about 5 minutes to about 96 hours; about 1 hour to about 72 hours; about 4 hours to about 32 hours, about 5 minutes to about 48 hours, or about 5 minutes to about 1 hour.

38. The method according to claim 35, wherein the skin or skin substitute is exposed to the ultraviolet radiation source at a minimal erythemal dose (MED).

39. The method according to claim 38, wherein the skin or skin substitute is exposed to the ultraviolet radiation source at a minimal erythemal dose (MED) of from about 1 MED to about 4 MED.

40. The method according to claim 35, wherein the skin or skin substitute is exposed to the ultraviolet radiation source at a minimal erythemal dose (MED) of from about 1 MED to about 4 MED for from about 5 minutes to about 48 hours.

41. The method according to claim 35, wherein the method is performed by an assay selected from the group consisting of microarray, Northern Blotting, polymerase chain reaction, reverse polymerase chain reaction, serial analysis of gene expression, and differential display.

42. The method according to claim 35, wherein said steps are repeated (i) over prolonged times of exposure to the ultraviolet radiation source, or (ii) for different times of exposure to the ultraviolet radiation source.

43. A method of preventing photoaged and/or photodamaged skin, comprising applying to the skin one or more ingredients in a concentration effective to modulate an alteration in expression level of at least one gene of a set of marker genes selected from the group consisting of Ras related protein RAB-7; Corneodesmosin; Amphiregulin; Granulocyte Chemotatic Protein; Migration inhibitory factor MRP8 (calgranulin A); Migration inhibitory factor MRP14 (calgranulin B); Ephrin receptor; epithelial cell kinase (ECK); shb proto-oncogene; MAD transcriptional repressor; Calpain; Leukocyte elastase inhibitor (monocyte/neutrophil elastase inhibitor); Placental plasminogen activator inhibitor (PAI-2); Beta-defensin (human beta defensin 2, HBD2 and human beta defensin 3, HBD3); Alpha 1 antitrypsin precursor; Tristetraproline; growth factor inducible nuclear protein 475; Interferon regulatory factors (IFR family); Nuclear Factor 1; hSNF2 Transcriptional activator; Prothymosin; GATA3 transcription factor; Histidine decarboxylase; Acyl Co-A binding protein; Decorin; CD44 antigen; B94 protein; Transthyretin (TTR), (prealbumin); Apolipoprotein E; Epithelial discoidin receptor; Thrombin receptor; serine/threonine protein phosphatase; Leukocyte antigen-related protein (LAR); Cytochrome p450 IVB1; Thioredoxin peroxidase; Myristylated Alanine-Rich C Kinase Substrate, MacMARCKS (MRP); and EB1 microtubule associated protein, wherein the one or more ingredients is applied to the skin for an effective period of time.

44. The method according to claim 43, wherein the period of time is selected from daily or from about 1 to 4 weeks.

45. The method according to claim 43, wherein photoaging of skin is treated, prevented, or ameliorated.

46. The method according to claim 43, wherein photodamage of skin is treated, prevented, or ameliorated.

47. A method of identifying or screening for individuals who are susceptible or highly sensitive to photodamage or photoaging of skin following exposure to UV radiation, comprising
(a) exposing to UV radiation a skin sample from an individual undergoing testing or screening; and
(b) determining if the expression of at least one gene of a set of marker genes is altered in the UV radiation exposed skin compared with a control (i) not exposed to UV radiation, or (ii) whose exposure to UV radiation is blocked or attenuated; said marker genes selected from the group consisting of Ras related protein RAB-7; Corneodesmosin; Amphiregulin; Granulocyte Chemotatic Protein; Migration inhibitory factor MRP8 (calgranulin A); Migration inhibitory factor MRP14 (calgranulin B); Ephrin receptor; epithelial cell kinase (ECK); shb proto-oncogene; MAD transcriptional repressor; Calpain; Leukocyte elastase inhibitor (monocyte/neutrophil elastase inhibitor); Placental plasminogen activator inhibitor (PAI-2); Beta-defensin (human beta defensin 2, HBD2 and human beta defensin 3, HBD3); Alpha 1 antitrypsin precursor; Tristetraproline; growth factor inducible nuclear protein 475; Interferon regulatory factors (IFR family); Nuclear Factor 1; hSNF2 Transcriptional activator; Prothymosin; GATA3 transcription factor; Histidine decarboxylase; Acyl Co-A binding protein; Decorin; CD44 antigen; B94 protein; Transthyretin (TTR), (prealbumin); Apolipoprotein E; Epithelial discoidin receptor; Thrombin receptor; serine/threonine protein phosphatase; Leukocyte antigen-related protein (LAR); Cytochrome p450 IVB1; Thioredoxin peroxidase; Myristylated Alanine-Rich C Kinase Substrate, MacMARCKS (MRP); EB1 microtubule associated protein; a unique subsequence of the at least one marker gene, and combinations thereof; wherein a determination of an alteration in gene expression of at least one of the marker genes identifies the individual as being susceptible or highly sensitive to photodamage or photoaging of skin.

48. A kit for assessing anti-photodamage or antiphotoaging properties of a substance, comprising:
a support material comprising a marker gene set selected from at least one of the group consisting of Ras related protein RAB-7; Corneodesmosin; Amphiregulin; Granulocyte Chemotatic Protein; Migration inhibitory factor MRP8 (calgranulin A); Migration inhibitory factor MRP14 (calgranulin B); Ephrin receptor; epithelial cell kinase (ECK); shb proto-oncogene; MAD transcriptional repressor; Calpain; Leukocyte elastase inhibitor (monocyte/neutrophil elastase inhibitor); Placental plasminogen activator inhibitor (PAI-2); Beta-defensin (human beta defensin 2, HBD2 and human beta defensin 3, HBD3); Alpha 1 antitrypsin precursor; Tristetraproline; growth factor inducible nuclear protein 475; Interferon regulatory factors (IFR family); Nuclear Factor 1; hSNF2 Transcriptional activator; Prothymosin; GATA3 transcription factor; Histidine decarboxylase; Acyl Co-A binding protein; Decorin; CD44 antigen; B94 protein; Transthyretin (TTR), (prealbumin); Apolipoprotein E; Epithelial discoidin receptor; Thrombin receptor; serine/threonine protein phosphatase; Leukocyte antigen-related protein (LAR); Cytochrome p450 IVB1; Thioredoxin peroxidase; Myristylated Alanine-Rich C Kinase Substrate, MacMARCKS (MRP); EB1 microtubule associated protein; a unique subsequence of the at least one marker gene, and combinations thereof; the at least one marker gene affixed to the support material;
and optionally comprising, nucleic acid probes, detection label, buffer, controls and instructions for use.

49. The kit according to claim 48, wherein the support material is selected from a nitrocellulose membrane, nylon membrane, plastic film, or glass slide.

50. The kit according to claim 49, wherein the assay performed by the kit is a microarray.
